(19) United States
(12) Patent Application Publication
(10) Pub. No.: US 2022/0202671 A1
(43) Pub. Date: Jun. 30, 2022

(54) METAL SEQUESTERING COSMETIC COMPOSITION

(71) Applicant: L'OREAL, Paris (FR)

(72) Inventors: Nicholas David STEBBINS, Clark, NJ (US); Patricia BRIEVA, Manalapan, NJ (US); Susan HALPERN, Basking Ridge, NJ (US); Stephen Matthew LYNCH, Westfield, NJ (US); Rabije CEKOVIC, Staten Island, NY (US)

(73) Assignee: L'OREAL, Paris (FR)

(21) Appl. No.: 17/125,945

(22) Filed: Dec. 17, 2020

(51) Int. Cl. A61K 8/365; A61K 8/60; A61K 8/67; A61K 8/34; A61K 8/02; A61Q 19/00; A61Q 19/10; A61Q 1/14; A61K 8/73; A61K 8/49; A61K 8/42; A61Q 17/04; A61K 8/368; A61K 8/41

(52) U.S. Cl. CPC A61K 8/365; A61K 2800/524; A61K 8/676; A61K 8/345; A61K 8/0291; A61Q 19/007; A61Q 19/10; A61Q 1/14; A61K 8/678; A61K 8/735; A61K 8/4946; A61K 8/42; A61Q 17/04; A61K 8/368; A61K 8/60; A61K 8/41; A61K 2800/48; A61K 2800/51; A61K 8/602

(57) ABSTRACT

A cosmetic composition including a metal chelation system includes a combination of ascorbic acid, ferulic acid and phytic acid in a carrier system that includes water, and further includes one or more other ingredients for providing one of a variety of forms of cosmetic products. The cosmetic composition demonstrates removal of free metal ions in material contacted with the cosmetic composition, the removal of free metal ions in a range from at least about 40% and up to about 99%.

### FIELD

This invention relates to a cosmetic composition for keratinous tissue, particularly skin, that includes a metal chelation system in a water-based carrier, that demonstrates highly efficient metal chelation/sequestering effective to minimize free radical formation.

### BACKGROUND

UV exposure and environmental pollution can accelerate skin aging by producing free radicals in skin. Antioxidants, including Vitamin C, and other actives protect cells from the damage of oxidative stress by scavenging free radicals and inhibiting oxidation reactions. The topical application of antioxidants and other skin actives is broadly employed in skin care products to prevent skin aging. Antioxidants are known to exhibit chemical properties that are associated with reduced tissue damage. Antioxidants are known to participate in sequestering chemically active species to prevent the formation of free radicals which can contribute to oxidative stress leading to aging of skin. There remains a need for enhanced compositions that demonstrate greater efficiency in sequestering chemically reactive species, for example metal species that adversely impact biological pathways through free radical formation.

### SUMMARY

The disclosure relates to an antioxidant system and a cosmetic composition comprising the antioxidant system that that demonstrates high efficiency in sequestering metal ions. The antioxidant system includes a combination of vitamin C, ferulic acid, and phytic acid combined in a water based system.

In some embodiments, the disclosure provides a cosmetic composition comprising:
(a) a metal chelation system comprising:
   i. ferulic acid; and
   ii. phytic acid;
(b) ascorbic acid; and
(c) a carrier system comprising a water phase.

In various embodiments, the cosmetic composition demonstrates one or both metal chelation and metal oxidation of at least one species of metal ions. In some specific examples the metal ions that are chelated or oxidized are selected from the group consisting of iron and copper. In some embodiments the cosmetic composition demonstrates one or both metal chelation and metal oxidation of one or a combination of metal ions. In some particular embodiments, the cosmetic composition demonstrates metal chelation or metal oxidation of iron and copper, each characterized by removal of free metal ions in material contacted with the cosmetic composition, the removal of free metal ions in a range from at least about 40% and up to about 99% removal of metal ions.

In various embodiments, the cosmetic composition is formed by providing the carrier system having a water-phase that comprises water. In various embodiments, the carrier system may further comprise one more additional ingredients, each present in the water phase or in another phase, such as an oily phase. In various embodiments, the one or more additional ingredients includes one or more of a glycol, a humectant, a preservative, a surfactant, a thickener, an active compound, or a combination thereof. In some embodiments, the cosmetic composition may include one or more of any one of the additional ingredients. In some embodiments, the cosmetic composition may also include emulsifiers, emollients, fragrances, pH adjusters, other cosmetically acceptable additives, or a combination thereof.

The cosmetic composition may be any suitable form, such as, but not limited to, a single phase solution comprising the water phase, a multi-phase solution comprising at least one water phase, an emulsion comprising a water phase, such as, but not limited to, a water-in-oil emulsion, an oil-in-water emulsion, or a silicone-in-water emulsion. The cosmetic composition may be provided in a product form for application to keratinous tissue, such as, but not limited to, a skin care product or a hair care product, and may be a product form such as, but not limited to, a hydroglycolic solution, a micellar water, a cream or lotion, a toner, a cleanser, or a make-up remover. The cosmetic composition in any of the product forms may be either a leave-on or a rinse-off formulation.

In various embodiments, the ascorbic acid is present in the cosmetic composition in a range from about 5% to about 30%, by weight, based on the weight of the composition, the ascorbic acid present in a ratio of water to ascorbic acid in a range from about 3:1 to about 10:1, based on the weight of the cosmetic composition.

In various embodiments, the ferulic acid is present in the cosmetic composition in a range from about 0.2% to about 5%, by weight, based on the weight of the composition.

In some embodiments, the cosmetic composition comprises phytic acid present in the cosmetic composition in a range from about 0.25% to about 10%, by weight, based on the weight of the composition.

In some embodiments, the disclosure provides a cosmetic composition comprising:
(a) a metal chelation system comprising:
   i. ferulic acid; and
   ii. phytic acid, chlorogenic acid or a combination thereof;
(b) ascorbic acid; and
(c) a carrier system comprising a water phase.

In some embodiments, the disclosure provides a cosmetic composition comprising:
(a) a metal chelation system comprising:
   i. ferulic acid; and
   ii. phytic acid;
(b) ascorbic acid; and
(c) a carrier system comprising: a water phase, and one or more additional components comprising a glycol, a humectant, a preservative, a surfactant, a thickener, an active compound, an emulsifier, an emollient, or a combination thereof.

In some embodiments, the disclosure provides a cosmetic composition comprising:
(a) a metal chelation system comprising:
   i. ferulic acid present in a range from about 0.2% to about 5%;
   ii. phytic acid present in a range from about 0.25% to about 10%;
(b) ascorbic acid present in a range from about 5% to about 30%; and
(c) a carrier system comprising a water phase;
all amounts by weight, based on the weight of the composition.

In some embodiments, the cosmetic composition comprises one or a combination of active compounds selected from the group consisting of Vitamin E, carnosine, hyaluronic acid, panthenol, at least one sun filter, at least one hydroxy acid, and combinations thereof. In some embodiments, an active compound is selected from the group consisting of alpha, beta or polyhydroxy acids selected from the group consisting of lactic acid, glycolic acid, salicylic acid, malic acid, tartaric acid, citric acid, mandelic acid, lactobionic acid, gluconolactone, galactose, and combinations thereof.

In some embodiments, the cosmetic composition is formed by providing the carrier system having a water-phase that comprises water and a glycol or combination of glycols selected from the group consisting of: dipropylene glycol, pentylene glycol, hexylene glycol, ethoxydiglycol, a combination of dipropylene glycol and pentylene glycol, and a combination of dipropylene glycol and hexylene glycol. In such embodiments, the cosmetic composition comprises water in a range from about 40% up to about 75%, based on the weight of the cosmetic composition.

In various embodiments, the cosmetic composition further includes one or more additives selected from emulsifiers, emollients, fragrances, pH adjusters, and other cosmetically acceptable additives, or a combination thereof, and is free or essentially free of powders and solid particles, including but not limited to, titanium dioxide, zinc oxide, tin oxide, iron oxides, mica, silica, ferric ferrocyanide, alumina, silicates, synthetic fluorphlogopite, polyethylene, polypropylene, poly methyl methacrylate, talc, perlites, hectorites, bentonite, kaolin, pumice, boron nitride, and combinations thereof. In some particular embodiments, the cosmetic composition excludes propylene glycol or butylene glycol.

These and other aspects of the invention are set out in the appended claims and described in greater detail in the detailed description of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

It is to be understood that both the foregoing general description and the following detailed description are exemplary and explanatory only and are not restrictive of the disclosure.

### DETAILED DESCRIPTION

“Cosmetically acceptable” means a carrier that is compatible with any keratinous substrate.

“Keratinous substrate” and “keratinous tissue” each includes but is not limited to skin, hair, and nails.

In various embodiments, the cosmetic composition includes a metal chelation system comprising the combination of ascorbic acid, ferulic acid and phytic acid in a carrier system that includes water, and further includes one or more other ingredients for providing one of a variety of forms of cosmetic products, the ingredients including but not limited to a glycol, a humectant, a preservative, a surfactant, a thickener, an active compound, or a combination thereof. The cosmetic composition may also include emulsifiers, emollients, fragrances, pH adjusters, other cosmetically acceptable additives, or a combination thereof. For the avoidance of doubt, the cosmetic composition may include one or more of any one of the additional ingredients. For example, the cosmetic composition may include one, or more than one glycol, or one, or more than one humectant, and the like. It will be understood that additional ingredients, when present, are present in the carrier system in either a water phase or an oily phase.

The cosmetic composition may be any suitable cosmetic composition, such as, but not limited to, a single phase solution comprising the water phase, a multi-phase solution comprising at least one water phase, an emulsion comprising a water phase, such as, but not limited to, a water-in-oil emulsion, an oil-in-water emulsion, or a silicone-in-water emulsion. The cosmetic composition may be provided in a product form for application to keratinous tissue, such as, but not limited to, a skin care product or a hair care product, and may be a product form such as, but not limited to, a hydroglycolic solution, a micellar water, a cream or lotion, a toner, a cleanser, or a make-up remover. The cosmetic composition in any of the product forms may be either a leave-on or a rinse-off formulation.

In some embodiments, the cosmetic composition according to the disclosure is a single phase solution that comprises one or more solutes dissolved in one or more solvents to form a substantially homogeneous liquid that appears to be substantially clear to the naked eye. In one such embodiment, the cosmetic composition is a single phase water solution. In some particular embodiments, the cosmetic composition is a hydroglycolic solution that comprises one or a combination of glycols

The inventors have shown the surprising and unexpected effect of the combination of ascorbic acid, ferulic acid and phytic acid as a potent metal chelator/sequester. Using this combination, the ability to chelate different metal ions such as iron and copper was increased in representative cosmetic composition comprising a hydroglycolic solution. The combination of ascorbic acid, ferulic acid and phytic acid demonstrated to the most chelation versus any other combination. Through a combination of oxidation and coordination, the level of copper and iron (II) was kept to a minimum in the cosmetic composition to diminish or eliminate oxidative reacting catalysis by the metal ions and thereby minimize oxidative stress in treated keratinous tissue.

In various embodiments, the cosmetic composition demonstrates one or both metal chelation and metal oxidation of at least one species of metal ions. In some embodiments the cosmetic composition demonstrates one or both metal chelation and metal oxidation of one or a combination of metal ions. In some particular embodiments, the cosmetic composition demonstrates metal chelation or metal oxidation of iron and copper, each characterized by removal of free metal ions in material, for example a fluid or a keratinous substrate, contacted with the cosmetic composition, the removal of free metal ions in a range from at least about 40% and up to about 99% removal of metal ions.

Metal Chelation System and Optional Ascorbic Acid

In accordance with the disclosure, embodiments of the cosmetic composition include a metal chelation system comprising a combination of ferulic acid, and phytic acid. In some embodiments, the chelation system comprises ferulic acid and one or both of phytic acid and chlorogenic acid. In some particular embodiments, the cosmetic composition includes in the chelation system each of ferulic acid and phytic acid. In yet other embodiments, the cosmetic composition comprises each of ascorbic acid, ferulic acid, and phytic acid. In addition to the metal chelation system, in some embodiments the cosmetic composition may include ascorbic acid (Vitamin C).

In some embodiments, pH of the cosmetic composition in the range from about 3.0 to about 4.0, and more particularly about 3.5, ensures that greater than about 82% of the ascorbic acid remains in a protonated, uncharged form as disclosed in U.S. Pat. No. 5,140,043, Aug. 18, 1992, the entire disclosure of which is incorporated by reference herein. Ascorbic acid may be provided by the addition of any reducing analog of ascorbic acid, such as D-isoascorbic acid or by the addition of other small reducing compounds such as, but not limited to, glutathione, L-cysteamine, and the like. Such forms would be expected to provide an equivalent composition to that claimed and are within the scope of the invention.

In the various embodiments, the cosmetic composition comprises ferulic acid, which is a cinnamic acid derivative. A cinnamic acid or derivative thereof includes ferulic acid, p-coumaric acid, caffeic acid, sinapinic acid, chlorogenic acids, caftaric acid, chicoric acid, coutaric acid, rosmarinic acid, derivatives thereof, and combinations thereof. Equivalent derivatives thereof include those cinnamic acid derivatives having substitutions on the hydroxyl groups of the aromatic ring such as short chain aliphatic groups (one to six carbon atoms) or long chain aliphatic groups (seven to twenty-four carbon atoms) to form an ether, or such aliphatic groups substituted with alkyl, alkoxy, hydroxyl, amino, or amido, for example, to form a substituted ether. Equivalent derivatives thereof further include those cinnamic acid derivatives having modifications of the methoxy group(s) of the aromatic ring to short chain aliphatic groups (two to six carbon atoms) or to long chain aliphatic groups (seven to twenty-four carbon atoms) to form a longer chain ether, or such aliphatic groups substituted with alkyl, alkoxy, hydroxyl, amino, or amido, for example, to form a substituted long chain ether. The 3-carboxy group of a cinnamic acid derivative may also be converted to esters or amides having aliphatic groups of up to 24 carbons or an aromatic group, for example. Cis and trans isomers of the cinnamic acid derivatives are included herein since the cis isomer is readily converted to the trans isomer. Salts of the cinnamic acid derivatives are included herein. In one embodiment, the cinnamic acid derivative is a triethanolamine salt. Caffeic acid, also known as 3-(3,4-dihydroxyphenyl)-2-propenoic acid, is found in many fruits, vegetables, seasonings and beverages consumed by humans. Caffeic acid is present in such goods in conjugated forms such as chlorogenic acid. Para-coumaric acid, also known as 3-(4-hydroxyphenyl)-2-propenoic acid or p-hydroxycinnamic acid, is found in various plants, including lignin forming plants. Trans-ferulic acid, also known as 3-(4-hydroxy-3-methoxyp-henyl)-2-propenoic acid or 4-hydroxy-3-methoxycinnamic acid, is also widely distributed in small amounts in plants. Sinapinic acid, also known as 3,5-dimethoxy4-hydroxycinnamic acid, is from black mustard seeds. Caffeic acid, para-coumaric acid, trans-ferulic acid and sinapinic acid are commercially available from Sigma-Aldrich.

In various embodiments, the cosmetic composition comprise at least one or a combination of phytic acid and chlorogenic acid. In some particular embodiments, the cosmetic composition comprise phytic acid. Phytic acid is also known as phytate and is a six-fold dihydrogenphosphate ester of inositol (specifically, of the myo isomer), also called inositol hexakisphosphate (IP6) or inositol polyphosphate. At physiological pH, the phosphates are partially ionized, resulting in the phytate anion. Phytic acid is found naturally in plant seeds and is a storage form of phosphorus. In some other embodiments, the cosmetic composition comprise chlorogenic acid. Chlorogenic acid is also known as chlorogenate or 3-caffeoylquinate and belongs to the class of organic compounds known as quinic acids and derivatives. Chlorogenic acid is the ester of caffeic acid (a hydroxycinnamic acid related to ferulic acid, as described herein above) and (−)-quinic acid.

In some embodiments the composition further includes ascorbic acid.

In various embodiments, each skin active in the at least one or a combination of skin actives comprising at least one or a combination of ascorbic acid, a cinnamic acid derivative, and Vitamin E, the combination present in the cosmetic composition at a concentration, from about 15% to about 30%, and in some embodiments, from about 16% to about 30%, and in some embodiments, from about 16% to about 25%, and in some embodiments, from about 16.5% to about 24%, or any suitable combination, sub-combination, range, or sub-range thereof by weight, based on the weight of the cosmetic composition.

Cinnamic acid derivatives, including, but not limited to ferulic acid, and triethanolamine salts may be present in the cosmetic compositions in an amount from about 0.1% to about 5%, and in some embodiments, from about 0.1% to about 3%, and in some embodiments, from about 0.5% to about 1.0%, or any suitable combination, sub-combination, range, or sub-range thereof by weight, based on the weight of the cosmetic composition.

Phytic acid may be present in the cosmetic compositions in an amount from about 0.5% to about 5%, and in some embodiments, from about 1% to about 4%, and in some embodiments, from about 1% to about 2%, or any suitable combination, sub-combination, range, or sub-range thereof by weight, based on the weight of the cosmetic composition. As exemplified in the instant disclosure, the phytic acid raw material is provided at a dilution of 50% such that the exemplified weight percents in the cosmetic compositions are multiplied by 0.5 to obtain the final weight percent of phytic acid.

Chlorogenic acid may be present in the cosmetic compositions in an amount from about 0.5% to about 1.5%, and in some embodiments, from about 1% to about 1.5%, and in some embodiments, from about 1.1% to about 1.2%, or any suitable combination, sub-combination, range, or sub-range thereof by weight, based on the weight of the cosmetic composition.

Ascorbic acid and its derivatives may be present in the cosmetic composition in an amount from about 1% to about 30%, and in some embodiments, from about 5% to about 25%, and in some embodiments, from about 10% to about 20%, and in some embodiments, from about 10% to about 15%, or any suitable combination, sub-combination, range, or sub-range thereof by weight, based on the weight of the cosmetic composition.

Phytic acid, carnosine, chlorogenic acid, hyaluronic acid, panthenol, may be present in the cosmetic composition in an amount from about 0.1% to about 5%, and in some embodiments, from about 0.1% to about 3%, and in some embodiments, from about 0.5% to about 1.0%, or any suitable combination, sub-combination, range, or sub-range thereof by weight, based on the weight of the cosmetic composition.

Thus, in various embodiments, each of the components of the metal chelation system and optionally ascorbic acid is present in a composition according to the disclosure, and each of the individual components in the ranges as described herein above, from about 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28 to about 30 percent by weight, including increments and ranges there between.

In various embodiments, the cosmetic composition demonstrates one or both metal chelation and metal oxidation of at least one species of metal ions. In some specific examples the metal ions that are chelated or oxidized are selected from the group consisting of iron and copper. In some embodiments the cosmetic composition demonstrates one or both metal chelation and metal oxidation of one or a combination of metal ions.

In some particular embodiments, the cosmetic composition demonstrates metal chelation or metal oxidation of iron and copper, each characterized by removal of free metal ions in material contacted with the cosmetic composition, the removal of free metal ions in a range from at least about 40% and up to about 99% removal of metal ions.

In some particular embodiments it is observed that an antioxidant solution of either ferulic acid or phytic acid, to which metal is added in a ratio of 1:1 and 1:10, metal to antioxidant, provides a removal of metal ions characterized by clustering/removal of free metal ions. In some examples, as shown herein, phytic acid helps diminish free radicals in a fluid or on a keratinous substrate by chelating metal, particularly iron ions, and ferulic acid help helps diminish free radicals in a fluid or on a keratinous substrate by chelating metal, particularly copper ions. In some particular embodiments, as exemplified herein, phytic acid chelates/removes metal, particularly iron, to thereby diminish the amount of iron ions by about 60%, and by about 80%, thereby leaving residual iron of about 20% and about 40% in respective examples. And according to such exemplified embodiments, ferulic acid chelates/removes metal, particularly copper, to thereby diminish the amount of copper ions by about 60% and by about 90%, thereby leaving residual copper in in an amount of about 10% and about 40% in respective examples.

Thus, in various embodiments, the cosmetic composition comprising a metal chelation system demonstrates chelation or removal of one or a combination of metal ions in a fluid or on a keratinous substrate in a range from about 40% to about 99%, or from about 40% to about 90% or from about 40% to about 80%, or from about 80% to about 90%, or from about 40, 41, 42, 43, 44, 45, 46, 47, 48, 49, 50, 55, 60, 65, 70, 75, 80, 85, 90, 95 to about 99 percent, including increments and ranges there between.

Carrier System

In various embodiments, the cosmetic composition is formed by providing a carrier system having a water-phase that comprises water.

Water

In accordance with the various embodiments, water is present in the cosmetic compositions in a range from about 10% to about 95%, and in some embodiments, from about 25% to about 80%, and in some embodiments, from about 40% to about 65%, and in some embodiments, from about 55% to about 60%, or any suitable combination, sub-combination, range, or sub-range thereof by weight, based on the weight of the cosmetic composition. In accordance with the some particular embodiments wherein the cosmetic composition is a hydroglycolic solution, water is present in the cosmetic compositions in a range from about 40% to about 75%, and in some embodiments, from about 45% to about 70%, and in some embodiments, from about 50% to about 65%, and in some embodiments, from about 55% to about 60%, and in some embodiments, about 68% or any suitable combination, sub-combination, range, or sub-range thereof by weight, based on the weight of the cosmetic composition. One of ordinary skill in the art, however, will appreciate that other ranges are within the scope of the invention. Thus, water is present, by weight, based on the total weight of the cosmetic composition, from about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, to about 75 percent, by weight, including increments and ranges therein and there between. One of ordinary skill in the art, however, will appreciate that other ranges are within the scope of the invention.

The water used may be chosen from, for example, sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of the cosmetic composition is not limited but is generally between 3.0 and 4.0, and in some embodiments, is one of between 3 and 3.5. The pH can be adjusted to the desired value by addition of a base (organic or inorganic) to the cosmetic composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid. It will be understood by a skilled artisan that pH is a measurement of the hydrogen ion concentration in water which is determined by measuring the electrode potential using electrodes attached to a pH meter. It will be further understood that, due to the intrinsic variability in pH measurements, a skilled artisan would expect at least a 10% variability in pH measurements of compositions such as disclosed herein when measured in a typical laboratory by a typical skilled person using a typical pH meter.

Other Ingredients

In various embodiments, the cosmetic composition also comprises one more additional ingredients, such as, but not limited to, a glycol, a humectant, a preservative, a surfactant, a thickener, an active compound, or a combination thereof. For the avoidance of doubt, the cosmetic composition may include one or more of any one of the additional ingredients. For example, the cosmetic composition may include one, or more than one glycol, or one, or more than one humectant, and the like. It will be understood that additional ingredients, when present, are present in the carrier system in either a water phase or an oily phase.

The cosmetic composition may be any suitable cosmetic composition, such as, but not limited to, a single phase solution comprising the water phase, a multi-phase solution comprising at least one water phase, an emulsion comprising a water phase, such as, but not limited to, a water-in-oil emulsion, an oil-in-water emulsion, or a silicone-in-water emulsion. The cosmetic composition may be provided in a product form for application to keratinous tissue, such as, but not limited to, a skin care product or a hair care product, and may be a product form such as, but not limited to, a hydroglycolic solution, a micellar water, a leave-on cream or lotion, a toner, a cleanser, or a make-up remover.

In some embodiments, the cosmetic composition comprises one or a combination of active compounds selected from the group consisting of Vitamin E, carnosine, hyaluronic acid, panthenol, at least one sun filter, at least one hydroxy acid, and combinations thereof. In some embodiments, an active compound is selected from the group consisting of alpha, beta or polyhydroxy acids selected from the group consisting of lactic acid, glycolic acid, salicylic acid, malic acid, tartaric acid, citric acid, mandelic acid, lactobionic acid, gluconolactone, galactose, and combinations thereof.

In various embodiments, the cosmetic composition further includes one or more additives selected from emulsifiers, emollients, fragrances, pH adjusters, other cosmetically acceptable additives, or a combination thereof, and is free or essentially free of powders and solid particles, including but not limited to, titanium dioxide, zinc oxide, tin oxide, iron oxides, mica, silica, ferric ferrocyanide, alumina, silicates, synthetic fluorphlogopite, polyethylene, polypropylene, poly methyl methacrylate, talc, perlites, hectorites, bentonite, kaolin, pumice, boron nitride, and combinations thereof. In some particular embodiments, the cosmetic composition excludes propylene glycol or butylene glycol.

Humectants/Glycols

In accordance with the disclosure, one or more humectants may be present in the cosmetic composition.

In some embodiments, the humectant may comprise one or more of polyols, including, for example, hexylene glycol, dipropylene glycol, glycerin, glycerol, glycols, such as, caprylyl glycol, butylene glycol, propylene glycol, isoprene glycol, polyethylene glycol, monoethylene glycol, diethylene glycol, triethylene glycol, diethylene glycol, hexylene glycol, glycol ethers, such as, monopropylene, dipropylene and tripropylene glycol alkyl(C1-C4)ethers, squalane, triacetin, sugars, such as, glucose, xylitol, maltitol, sorbitol, sucrose pentaerythritol, inositol, pyrrolidone carboxylic acid, lactic acid, lithium chloride, acetamide MEA, sodium lactate, urea, dicyanamide, hyaluronic acid, aloe vera, honey, and seaweed extract.

In accordance with some embodiments, glycols, when present, may be present in the cosmetic composition alone or with at least one other glycol, each glycol present in an amount that is in a range from about 0.1% to about 30%, or from about 5% to about 25%, or from about 10% to about 15%, or any suitable combination, sub-combination, range, or sub-range thereof by weight, based on the weight of the composition, by weight, based on the weight of the cosmetic composition. In some embodiments, the total amount of glycol is less than about 30%, for example, the total is about 29.5%, or about 29%. As provided herein, some glycols, when present alone in the cosmetic composition, are present in an amount that is at least about 8%, or at least about 8.5%, or at least about 9%, or at least about 9.5%, or at least about 10%, or at least about 10.5%.

Thus, in various embodiments, each of the glycols is present in a cosmetic composition according to the disclosure, each alone and/or in combinations as described in the paragraphs set forth herein above from about 0.1, 0.2, 0.3, 0.4, 0.5, 0.6, 0.7, 0.8, 0.9, 1.0, 1.5, 2, 3, 4, 5, 6, 7, 8, 9, 10, 11, 12, 13, 14, 15, 16, 17, 18, 19, 20, 21, 22, 23, 24, 25, 26, 27, 28, 29 to less than about 30 percent, by weight, including increments and ranges therein and there between.

In some embodiments, the composition includes a glycol humectant selected from the group consisting of hexylene glycol, dipropylene glycol, pentylene glycol, propylene glycol, ethoxydiglycol, and combinations thereof. In some embodiments, the composition includes ethoxydiglycol, propylene glycol and pentylene glycol, or hexylene glycol, pentylene glycol and dipropylene glycol, or dipropylene glycol, or hexylene glycol and dipropylene glycol. In some embodiments, the composition includes glycerin.

In accordance with the various embodiments, the amount of humectant present in the cosmetic composition can range from about 1% to about 10%, or from about 1% to about 8%, or from about 1% to about 5%, or from about 2% to about 3%, or any suitable combination, sub-combination, range, or sub-range thereof by weight, based on the weight of the composition. One of ordinary skill in the art, however, will appreciate that other ranges are within the scope of the invention.

Thus, any one of or a combination of humectant, when present, may be present, by weight, based on the total weight of the composition, is from about 1, 2, 3, 4, 5, 6, 7, 8, 9, to about 10 percent, by weight, based on the weight of the cosmetic composition, including increments and ranges therein and there between.

Preservatives

In accordance with the disclosure, one or more preservatives and/or antimicrobials may be present in the composition. Any preservative commonly used in cosmetic formulations is an acceptable preservative for the cosmetic compositions herein, such as phenoxyethanol, members from the paraben family such as the methyl, ethyl, propyl, butyl or isobutyl parabens, 4-hydroxy benzoic acid, benzoic acid, sorbic acid, dehydroacetic acid, triclosan, benzyl alcohol, chlorophenesin, or salicylic acid, for example. At more concentrated amounts of suitable solvents for optional additives, in particular, suitable solvents for antimicrobials and preservatives, members from the paraben family may be used as a preservative.

In some embodiments, the preservative may comprise one or more of preservatives selected from the group consisting of organic acids, parabens, formaldehyde donors, phenol derivatives, quaternary ammoniums, alcohols, isothiazolinones, and combinations thereof. Preservatives having antibacterial activity are optionally present in the cosmetic compositions of the present invention. Examples of organic acid preservatives include, but are not limited to, sodium benzoate, potassium sorbate, benzoic acid and dehydroacetic acid, sorbic acid, and combinations thereof. A preferred organic acid preservative system includes a mixture of sodium benzoate and potassium sorbate. Examples of paraben preservatives include, but are not limited to, alkyl para-hydroxybenzoates, wherein the alkyl radical has from 1, 2, 3, 4, 5 or 6 carbon atoms and for example, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Examples of formaldehyde donor preservatives include, but are not limited to, 1,3-DimethyloI-5,5-dimethylhydantoin (DMDM hydantoin), imidazolidinyl urea, gluteraldehyde, and combinations thereof. Examples of quaternary ammonium preservatives include, but are not limited to, benzalkonium chloride, methene ammonium chloride, benzethonium chloride, and combinations thereof. Examples of alcohol preservatives include, but are not limited to, ethanol, benzyl alcohol, dichlorobenzyl alcohol, phenoxyethanol, and combinations thereof. Examples of isothiazolone preservatives include, but are not limited to, methylchloroisothiazolinone, methylisothiazolinone, and combinations thereof.

Other suitable preservatives include, but are not limited to, chloracetamide, triclosan and iodopropynyl butylcarbamate, pyridine derivatives (e.g., pyrithione and zinc pyrithione), chlorphenesin, phenyl mercuric salts, phenoxyethanol, and other known preservative systems.

In some embodiments, the preservative includes one or more preservatives, the one or combination present at a concentration, by weight of about 0.001% to about 5%, or alternatively about 0.05% to about 2.5% or alternatively about 0.1% to about 2.0%, based upon weight of the composition.

Thus, any one of or a combination of preservatives, when present, may be present, by weight, based on the total weight of the composition, is from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2, 3, 4, to about 5 weight percent, including increments and ranges therein and there between.

Surfactant

In accordance with the disclosure, various non-limiting embodiments of the cosmetic composition may optionally include at least one surfactant. And in accordance with the disclosure, various non-limiting embodiments of the cosmetic composition that include an oil, for example, but not limited to, a Vitamin E component, such as tocopherol, may optionally include at least one surfactant. In some embodiments, the cosmetic composition comprises no oil. In some embodiments, the cosmetic composition comprises no surfactant. In some embodiments, the cosmetic composition comprises at least one oil, for example, tocopherol, with at least one surfactant. In some embodiments, the cosmetic composition comprises at least one oil with more than one surfactant. In some embodiments, the cosmetic composition comprises at least one oil without any surfactants, wherein the total amount of oil present does not inhibit forming the cosmetic composition as a clear, single phase solution.

The at least one surfactant may be an nonionic, cationic, anionic, or a zwitterionic surfactant. The at least one surfactant may be selected from the group consisting of polyoxyethylene sorbitan monolaureate, laureth-23, polyoxyethylated octyl phenol, 3-((3-cholamidopropyl) dimethylammonio)-1 propane sulfonate, sodium dilauramidoglutamide lysine, cholate, deoxycholate, sodium dodecylsulfate, TWEEN-80, and combinations thereof. The at least one surfactant may exclude esters. The at least one surfactant may be present in the cosmetic composition in a range from about 1% to about 5%, based on the weight of the cosmetic composition.

In some particular embodiments the at least one surfactant, when present, comprises one or a combination of polyoxyethylene sorbitan monolaureate and laureth-23.

In various embodiments, the at least one surfactant, when present, may be present from about 1% to about 5% by weight of the cosmetic composition, and in some embodiments, from about 1% to about 2%, and in some embodiments, from about 3% to about 6%, and in some embodiments, from about 3% to about 4.5%, or any suitable combination, sub-combination, range, or sub-range thereof by weight, based on the weight of the cosmetic composition. In some embodiments, laureth-23 may be present from about 1% to about 5% by weight of the cosmetic composition, and in some embodiments, from about 3% to about 4.5%, or any suitable combination, sub-combination, range, or sub-range thereof by weight, based on the weight of the cosmetic composition. In some embodiments, polyoxyethylene sorbitan monolaureate may be present from about 1% to about 5% by weight of the cosmetic composition, and in some embodiments, from about 1% to about 2%, or any suitable combination, sub-combination, range, or sub-range thereof by weight, based on the weight of the cosmetic composition. In some embodiments, the cosmetic composition comprises more than one surfactant. One of ordinary skill in the art, however, will appreciate that other ranges are within the scope of the invention.

Thus, in various embodiments, when present, each of the at least one surfactant may be present in a composition according to the disclosure from about 1, 2, 3, 4, to about 5 percent, by weight, including increments and ranges therein and there between.

Polymeric Thickener

In accordance with the various embodiments, the composition may include one or more polymeric thickeners. In some embodiments, the one or more thickener may be selected from one or more of natural gums and synthetic polymers, for example, the thickener may be selected from the group consisting of starches (corn, rice, tapioca, potato), gums (xanthan carrageenan, gellan, sclerotium, tarabiotech fermentation). In some particular embodiments, the thickener may be selected from acrylates/C10-30 alkyl acrylate crosspolymer, carbomer, xanthan gum, hydroxypropyl guar, ceratonia siliqua (carob) gum, ammonium polyacryloyldimethyl taurate, ammonium acryloyldimethyltaurate/steareth-25 methacrylate crosspolymer, and polyacrylate crosspolymer-6.

In some embodiments, the polymeric thickener may be one of carbomer, acrylates/C10-30 alkyl acrylate crosspolymer, polyacrylate crosspolymer-6, microcrystalline cellulose (and) cellulose gum, xanthan gum, sodium carboxymethyl starch, sclerotium gum (and) xanthan gum, xanthan gum (and) ceratonia siliqua (carob) gum (50/50), dehydroxanthan gum, hydroxypropyl starch phosphate, sclerotium gum (and) xanthan gum (75/25), sclerotium gum, xanthan gum (and) sclerotium gum (and) Lecithin (and) pullulan, or combinations thereof.

In some embodiments the composition may comprise two or more polymeric thickeners. The amount of each of the at least one polymeric thickener, when present, may be present in the composition in a range of from about 0.01% to about 2%, or from about 0.01% to about 1.5%, or from about 0.3% to about 1.2%, or any suitable combination, sub-combination, range, or sub-range thereof by weight, based on the weight of the composition. One of ordinary skill in the art, however, will appreciate that other ranges are within the scope of the invention.

In some embodiments, the total amount of polymeric thickener in the composition, when present, is present from about 0.01% to about 5%, or from about 0.02% to about 2%, or from about 0.03% to about 1.5%, or from about 0.1% to about 0.2%, or any suitable combination, sub-combination, range, or sub-range thereof by weight, based on the weight of the composition. One of ordinary skill in the art, however, will appreciate that other ranges are within the scope of the invention.

Thus, one or more polymeric thickener, when present, is present by weight, based on the total weight of the composition, from about 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5 to about 5.0 percent, including increments and ranges therein and there between.

Optional Active Compounds

In accordance with the disclosure, embodiments of the cosmetic composition may include one or more additional compounds selected from active compounds, in particular keratinous tissue actives, such as, but not limited to, skin actives. In some embodiments, optional active compounds may be selected from the group consisting of: vitamins, for example, Vitamin E, carnosine, hyaluronic acid, panthenol, at least one sun filter, at least one hydroxy acid, and combinations thereof. In some embodiments, an active compound is selected from the group consisting of alpha, beta or polyhydroxy acids selected from the group consisting of lactic acid, glycolic acid, salicylic acid, malic acid, tartaric acid, citric acid, mandelic acid, lactobionic acid, gluconolactone, galactose, and combinations thereof. For the avoidance of doubt, the cosmetic composition may include one or more of any one of the optional active compounds. For example, the cosmetic composition may include one, or more than one hydroxy acid, one, or more than one vitamin, and the like.

Vitamin E may be selected from the group consisting of alpha-tocopherol, beta-tocopherol, delta-tocopherol, gamma-tocopherol, and alpha-tocotrienol, beta-tocotrienol, delta-tocotrienol, gamma-tocotrienol, and derivatives thereof. Salts or derivatives of tocopherols include pharmaceutically acceptable compounds such as acetate, sulfate, succinate, nicotinate, palmitate, allophanate, phosphate, quinone, or halogenated derivatives, esters, or stereoisomers, for example. The invention encompasses the use of Vitamin E derivatives in which substitutions, additions, and other alterations have been made in the 6-chromanol ring and/or side chain, with the proviso that the derivatives maintain the antioxidant activity of Vitamin E. Additional tocopherols can be constructed by conjugation to the ring structure or side chain of various other moieties, such as those containing oxygen, nitrogen, sulfur and/or phosphorus. Tocopherol derivatives can also be made by modifying the length of the side chain from that found in tocopherols such as alpha-, beta-, delta- and gamma-tocopherol. Tocopherols can also vary in stereochemistry and saturation of bonds in the ring structure and side chain. Additional tocopherol derivatives, including prodrugs, can be made by conjugation of sugars or other moieties to the side chain or ring structure. Tocopherols include without limitation stereoisomers (e.g., + and − stereoisomers of alpha-tocopherol; (+/−) indicates a racemic mixture) or mixtures of structurally distinct tocopherols (e.g., alpha-plus gamma-tocopherol). Tocopherols may be obtained from Roche, Nutley, N.J., for example.

In some embodiments, additional actives can also include at least one hydroxy acid selected from alpha, beta or polyhydroxy acids. Thus, in various embodiments, a hydroxy acid may be selected from the group consisting of lactic acid, glycolic acid, salicylic acid, malic acid, tartaric acid, citric acid, mandelic acid, lactobionic acid, gluconolactone, galactose, and combinations thereof.

In some embodiments, additional actives can also include one or more of the antioxidants selected from the group consisting of mangiferin, baicalin, resveratrol, tannic acid, polyphenols, amino acids and derivatives thereof, imidazoles, carnosine derivatives, carotenoids, carotenes (such as α-carotene, β-carotene, and lycopene), vitamin A, co-enzyme Q10, bioflavonoids, glutathione, plant extracts (such as rosemary extract, olive leaf extracts), green tea extracts, and combinations thereof.

In some particular embodiments, the cosmetic composition includes a combination of active compounds present in the cosmetic composition comprising tocopherol, panthenol, hyaluronic acid, hydroxy acid, carnosine, or a combination thereof.

Generally, each of the optional active compounds is present in the cosmetic composition in an amount from about 0.1% to about 30%, and in some embodiments, from about 0.5% to about 30%, and in some embodiments, from about 0.5% to about 15%, and in some embodiments, from about 0.1% to about 1%, and in some embodiments, from about 1% to about 2%, or any suitable combination, sub-combination, range, or sub-range thereof by weight, based on the weight of the cosmetic composition.

Vitamin E and its derivatives, including, but not limited to tocopherol, may be present in the cosmetic compositions in an amount from about 0.5% to about 2%, and in some embodiments, from about 0.5% to about 1%, and in some embodiments, from about 1% to about 2.0%, or any suitable combination, sub-combination, range, or sub-range thereof by weight, based on the weight of the cosmetic composition.

Carnosine, hyaluronic acid, panthenol, may be present in the cosmetic compositions in an amount from about 0.1% to about 5%, and in some embodiments, from about 0.1% to about 3%, and in some embodiments, from about 0.5% to about 1.0%, or any suitable combination, sub-combination, range, or sub-range thereof by weight, based on the weight of the cosmetic composition.

Carnosine may be present in the cosmetic compositions in an amount from about 0.10% to about 1%, and in some embodiments, from about 0.20% to about 0.5%, and in some embodiments, from about 0.20% to about 0.25%, or any suitable combination, sub-combination, range, or sub-range thereof by weight, based on the weight of the cosmetic composition.

Hyaluronic acid may be present in the cosmetic compositions in an amount from about 0.01% to about 1%, and in some embodiments, from about 0.01% to about 0.1%, and in some embodiments, from about 0.05% to about 0.07%, or any suitable combination, sub-combination, range, or sub-range thereof by weight, based on the weight of the cosmetic composition.

Panthenol may be present in the cosmetic compositions in an amount from about 0.1% to about 1%, and in some embodiments, from about 0.1% to about 0.5%, and in some embodiments, from about 0.2% to about 0.4%, and in some embodiments, from about 0.2% to about 0.25%, or any suitable combination, sub-combination, range, or sub-range thereof by weight, based on the weight of the cosmetic composition.

A hydroxy acid may be present in the cosmetic compositions in an amount from about 0.25% to about 10%, and in some embodiments, from about 0.5% to about 8%, and in some embodiments, from about 1% to about 5%, and in some embodiments, from about 0.25% to about 0.5%, or any suitable combination, sub-combination, range, or sub-range thereof by weight, based on the weight of the cosmetic composition.

Thus, in various embodiments, optional active compounds, if present, are present in a composition according to the disclosure, and each of the individual components in the ranges as described herein above, from about 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28 to about 30 percent by weight, including increments and ranges there between.

Optional Additives

The compositions can also comprise at least one additive used in the cosmetics field which does not affect the properties of the compositions according to the invention, such as, emulsifiers, emollients, fragrances, pH adjusters (citric acid, sodium chloride; neutralizing or pH-adjusting agents (e.g., triethanolamine (TEA) and sodium hydroxide), and combinations thereof), other cosmetically acceptable additives, such as but not limited to, pearlescent agents, silica, and coloring materials; essential oils; fruit extracts, for example, Pyrus Malus (Apple) Fruit Extract, and Aloe Barbadensis Leaf Juice Powder. Although the optional additives are given as examples, it will be appreciated that other optional components compatible with cosmetic applications known in the art may be used.

In accordance with the various embodiments, the amount of one or more additives, alone or in combination, present in the composition can be present in the composition according to the disclosure in a range from about 0.001% to about 20%, by weight, or from about 0.005% to about 0.01%, or from about 0.01% to about 0.1%, or from about 0.15% to about 5%, or from about 0.40% to about 4%, or from about 0.5% to about 2.5% by weight, or from about 1% to about 2%, or any suitable combination, sub-combination, range, or sub-range thereof by weight, based on the total weight of the composition.

Thus, any one or a combination of additives may be present, by weight, based on the total weight of the composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and ranges therein and there between.

Although the optional additives are given as examples, it will be appreciated that other optional components compatible with cosmetic applications known in the art may be used that are suitable. It will be appreciated by a skilled artisan that any optional additives are present only to the extent and in amounts that do not materially adversely affect the basic and novel characteristic(s) of the claimed disclosure. Thus, in some embodiments that include optional additives, such optional additives will not materially adversely affect the solubility of the skin actives of the cosmetic composition. And in some embodiments that include optional additives, such optional additives will not materially adversely affect the cosmetic composition forming a single phase solution.

In some particular embodiments, the cosmetic composition may include optional additives, for example one or more of phenoxyethanol, terephthalylidene dicamphor sulfonic acid, trisodium ethylenediamine disuccinate, sodium citrate, sodium chloride, hydroxyacetophenone sodium benzoate, potassium sorbate, citric acid, caprylyl glycol, trisodium ethylenediamine disuccinate, or combinations thereof. In some embodiments, phenoxyethanol is present as a preservative in the cosmetic composition in a range from about 0.5% to about 2%, and in some embodiments in a range from about 1% to about 2%.

In accordance with the various embodiments, the amount of one or more actives and additives, alone or in combination, when present in the cosmetic composition according to the disclosure can be present in a range from about 0.001% to about 20%, and in some embodiments, from about 0.05% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5%, and in some embodiments, from about 1% to about 2%, or any suitable combination, sub-combination, range, or sub-range thereof by weight, based on the total weight of the cosmetic composition. One of ordinary skill in the art, however, will appreciate that other ranges are within the scope of the invention.

Thus, any one or a combination of actives and additives may be present, each one or the combination present from about 0.001, 0.005, 0.01, 0.02, 0.03, 0.04, 0.05, 0.06, 0.07, 0.08, 0.09, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 percent, by weight, including increments and ranges therein and there between.

Formulations

The cosmetic composition of the present invention may be used for the production of cosmetic preparations, or dermatological preparations, more particularly topical treatment preparations, that may be formulated as single-phase solution compositions, cosmetic serums, or aerosols, for example. Topical application to a surface may be a surface such as the mucus membrane or the skin, for example.

The examples below according to the invention are given by way of illustration and without a limiting nature. The names are the chemical name or the INCI name. The amounts of active ingredients are given therein as % by weight based on raw material compositions comprising 100% of the listed ingredient, unless otherwise mentioned.

### EXAMPLES

### Example 1: Inventive Compositions

Various representative embodiments of the inventive compositions and articles are exemplified herein.

TABLE 1
Inventive Compositions

| | | | | INV 4 |
|---|---|---|---|---|
| | | | | FERULIC + |
| | | | | (2% AM |
| | INV 1 | INV 2 | INV 3 | PHYTIC |
| | FERULIC + | FERULIC + | FERULIC + | ACID, |
| | (1% AM | (2% AM | (3% AM | 189N |
| | PHYTIC | PHYTIC | PHYTIC | GLYCOL |
| INCI UE | ACID) | ACID) | ACID) | BLEND) |
| GLYCERIN | 5 | 5 | 5 | |
| WATER | QS | QS | QS | QS |
| PHENOXY- | 1 | 1 | 1 | 1 |
| ETHANOL | | | | |
| ACTIVES | ~1.3 | ~1.3 | ~1.3 | ~1.3 |
| (PANTHENOL, | | | | |
| TOCOPHEROL, | | | | |
| SODIUM | | | | |
| HYALU- | | | | |
| RONATE) | | | | |
| FERULIC ACID | 0.5 | 0.5 | 0.5 | 0.5 |
| TRIETHANOL- | 0.5 | 0.5 | 0.5 | 0.5 |
| AMINE | | | | |
| LAURETH-23 | 3 | 3 | 3 | 3 |
| PHYTIC ACID | 2 | 4 | 6 | 4 |
| ASCORBIC | 15 | 15 | 15 | 15 |
| ACID | | | | |
| DIPROPYLENE | 20 | 20 | 20 | 15.71 |
| GLYCOL | | | | |
| HEXYLENE | | | | 9.29 |
| GLYCOL | | | | |

*Phytic Acid raw material = 50% phytic acid in carrier

TABLE 2
Inventive Compositions (cont)

| | INV 5 | | | |
|---|---|---|---|---|
| | FERULIC + | INV 6 | INV 7 | |
| | (2% AM | FERULIC + | FERULIC + | |
| | PHYTIC ACID, | (0.5% AM | (4% AM | |
| | 190N GLYCOL | PHYTIC | PHYTIC | INV 8 |
| INCI UE | BLEND) | ACID) | ACID) | BASE |
| GLYCERIN | | 5 | 5 | 5 |
| WATER | QS | QS | QS | QS |
| PHENOXY- | 1 | 1 | 1 | 1 |
| ETHANOL | | | | |
| FERULIC ACID | 0.5 | 0.5 | 0.5 | 0.5 |
| TRIETHANOL- | 0.5 | 0.5 | 0.5 | 0.5 |
| AMINE | | | | |
| LAURETH-23 | 3 | 3 | 3 | 3 |
| PHYTIC ACID | 4 | 1 | 8 | 2 |
| ASCORBIC ACID | 15 | 15 | 15 | 15 |
| DIPROPYLENE | 9.28 | 20 | 20 | |
| GLYCOL | | | | |
| HEXYLENE | ~15 | | | |
| GLYCOL | | | | |
| & PENTYLENE | | | | |
| GLYCOL | | | | |
| PROPYLENE | | | | ~25 |
| GLYCOL & | | | | |
| ETHOXY- | | | | |
| DIGLYCOL | | | | |

*Phytic Acid raw material = 50% phytic acid in carrier

TABLE 3
Summary of compositions

| | FERULIC | PHYTIC | ASCORBIC | |
|---|---|---|---|---|
| COMP | ACID | ACID | ACID | GLYCOLS |
| INV 1 | 0.5% | 2% | 15% | 20% DIPROPYLENE |
| | | | | GLYCOL |
| INV 2 | 0.5% | 4% | 15% | 20% DIPROPYLENE |
| | | | | GLYCOL |
| INV 3 | 0.5% | 6% | 15% | 20% DIPROPYLENE |
| | | | | GLYCOL |
| INV 4 | 0.5% | 4% | 15% | 16% DIPROPYLENE |
| | | | | GLYCOL |
| | | | | 9% HEXYLENE |
| | | | | GLYCOL |
| INV 5 | 0.5% | 1% | 15% | 9% DIPROPYLENE |
| | | | | GLYCOL |
| | | | | 5% PENTYLENE |
| | | | | GLYCOL |
| | | | | 11% HEXYLENE |
| | | | | GLYCOL |
| INV 6 | 0.5% | 8% | 15% | 20% DIPROPYLENE |
| | | | | GLYCOL |
| INV 7 | 0.5% | 2% | 15% | 20% DIPROPYLENE |
| | | | | GLYCOL |
| INV 8 | 0.5% | 2% | 15% | 5% PROPYLENE |
| | | | | GLYCOL |
| | | | | 20% ETHOXY- |
| | | | | DIGLYCOL |

**all compositions include other actives that include HA, VIT E + SURFACTANT, PANTHENOL, PRESERVATIVE, and other ingredients including GLYCERIN

### Example 2: Demonstrating Metal Chelation/Sequestering

Electron paramagnetic resonance (EPR) tests were carried out in which a solution of Fe(II) or Cu(II) was added to solutions of ferulic acid and phytic acid, both alone, in combination (ferulic+phytic), and with ascorbic acid (ferulic+phytic+ascorbic). The chelation/coordination of metals was observed over time.

In the tests, a 10 microliter of stock solutions (5000 or 500 ppm) of Fe(II) or Cu(II) was combined with 90 microliter phytic acid solution. EPR was measured vs time and colorimetric measurement was made to measure residual free metals.

With EPR testing, it was shown that ferulic acid chelates Cu(II) very well. When phytic acid was tested using EPR, it was determined that Fe(II) was strongly chelated. Additionally, phytic acid oxidized Fe(II) into Fe(III). In the presence of ascorbic acid and phytic acid, 90% of Fe(II) is oxidized into Fe(III) by ascorbic acid. The remaining Fe(II) is chelated and oxidized into Fe(III) by phytic acid. That is, the ascorbic acid, ferulic acid, and phytic acid association addresses Fe and Cu chelation for inhibiting any oxidative reacting catalysis by these transition metals. This chelation/oxidation is essential as Fe(III) generates fewer free radicals than Fe(II), with respect to biological pathways and oxidative stress.

In some particular embodiments it is observed that an antioxidant solution of either ferulic acid or phytic acid, to which metal is added in a ratio of 1:1 and 1:10, metal to antioxidant, provides a removal of metal ions characterized by clustering/removal of free metal ions. Referring now to Table 4, the results show reduction of metal ions by ferulic and phytic acids.

TABLE 4
Coordination or Reduction of Metal Ions by Phytic and Ferulic Acids

| | | | | % Coordinated | % Non-Coordinated |
|---|---|---|---|---|---|
| Ferulic | | Phytic | | or | or Non-Reduced |
| Acid | Iron | Acid | Copper | Reduced Metal | Metal |
| 1 | 1 | | | 0 | ~100 |
| 10 | 1 | | | 0 | ~100 |
| 1 | | | 1 | 60 | 40 |
| 10 | | | 1 | ~60 | ~40 |
| | 1 | 1 | | 90 | 10 |
| | 1 | 10 | | ~65 | ~35 |
| | | 1 | 1 | 80 (reduced) | 20 |
| | | 10 | 1 | >90 (reduced) | <10 |

* [symbol] [symbol]
indicates data missing or illegible when filed

As shown on Table 4, ferulic acid chelates copper to thereby diminish the amount of copper ions by about 60% at a ratio of 1:1 with the metal, and by about the same (60%) at a ratio of 10:1 (ferulic acid to metal). And phytic acid chelates to form aggregates with iron to thereby diminish the amount of iron ions by about 90% at a ratio of 1:1 with the metal, and by about 65% at a ratio of 10:1 (phytic acid to metal). The data further show that ferulic acid has no discernible effect on iron ions and phytic acid effectively reduced copper (II), but does not chelate it.

### Example 3: Metal Removal by Cosmetic Composition

In some particular embodiments it is observed that a cosmetic composition according to the disclosure and comprising both ferulic acid and phytic acid, provides a removal of metal ions added to the composition. Referring now to Table 5, the results show reduction of metal ions by ferulic and phytic acids as evidenced by measured residual metal ions.

TABLE 5
Metal Ions Removal in Exemplary Cosmetic Composition

| Composition: | Residual FE | Residual CU |
|---|---|---|
| Negative Control (no | 500 ppm | 500 ppm |
| ferulic or phytic acids) | | |
| Inventive 2 (includes | 100 ppm | 200 ppm |
| both ferulic and phytic | (reduced by 80%) | (reduced by 60%) |
| acids) | | |

As shown on Table 5, a composition according to the disclosure demonstrates removal of free metal ions of about 60% with respect to copper and about 80% of iron.

Together, each of tables 4 and 5 show that phytic clearly and unexpectedly chelates iron, but not copper and ferulic clearly and unexpectedly chelates copper, but not iron. These two acids together, in combination with Vit C, give the best overall result, as shown on Table 5.

While the disclosure has been described with reference to described embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the cosmetic composition, typically 0% by weight, based on the total weight of the cosmetic composition. The term “essentially free” means that, while it prefers that no excluded material is present in the cosmetic composition, it is possible to have very small amounts of the excluded material in the cosmetic composition of the invention, provided that these amounts do not materially affect the advantageous properties of the cosmetic composition. In particular, “essentially free” means that excluded material can be present in the cosmetic composition at an amount of less than about 0.1% by weight, based on the total weight of the cosmetic composition.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

The terms “weight percent” and “wt %” may be used interchangeably and mean percent by weight, based on the total weight of a composition, article or material, except as may be specified with respect to, for example, a phase, or a system that is a component of a composition, article or material. All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A cosmetic composition comprising:
(a) a metal chelation system comprising:
i. ferulic acid; and
ii. phytic acid;
(b) ascorbic acid; and
(c) a carrier system comprising a water phase.

2. The cosmetic composition according to claim 1, wherein the cosmetic composition demonstrates removal of free metal ions in material contacted with the cosmetic composition, the removal of free metal ions in a range from at least about 40% and up to about 99%.

3. The cosmetic composition according to claim 1, wherein the carrier system further comprises one more additional ingredients, each present in the water phase or in a second phase.

4. The cosmetic composition according to claim 3, wherein the second phase is an oily phase.

5. The cosmetic composition according to claim 3, wherein the one or more additional ingredients includes one or more of a glycol, a humectant, a preservative, a surfactant, a thickener, an active compound, or a combination thereof.

6. The cosmetic composition according to claim 1, wherein the cosmetic composition is one of a single phase solution comprising the water phase, a multi-phase solution comprising at least one additional phase, or an emulsion comprising an oily phase.

7. The cosmetic composition according to claim 1, wherein the cosmetic composition is a product for keratinous tissue selected from a hydroglycolic solution, a micellar water, a cream, a lotion, a toner, a cleanser, or a make-up remover.

8. The cosmetic composition according to claim 1, wherein the cosmetic composition a leave-on or a rinse-off formulation.

9. The cosmetic composition according to claim 1, wherein the ascorbic acid is present in the cosmetic composition in a range from about 5% to about 30%, by weight, based on the weight of the composition

10. The cosmetic composition according to claim 9, wherein the ascorbic acid present in a ratio of water to ascorbic acid in a range from about 3:1 to about 10:1, based on the weight of the cosmetic composition.

11. The cosmetic composition according to claim 1, wherein the ferulic acid is present in the cosmetic composition in a range from about 0.2% to about 5%, by weight, based on the weight of the composition.

12. The cosmetic composition according to claim 1, wherein phytic acid present in the cosmetic composition in a range from about 0.25% to about 10%, by weight, based on the weight of the composition.

13. The cosmetic composition according to claim 1, wherein: ferulic acid is present in a range from about 0.2% to about 5%; phytic acid is present in a range from about 0.25% to about 10%; and ascorbic acid is present in a range from about 5% to about 30% all amounts by weight, based on the weight of the composition.

14. The cosmetic composition according to claim 1, wherein the cosmetic composition comprises one or a combination of active compounds selected from the group consisting of Vitamin E, carnosine, hyaluronic acid, panthenol, at least one sun filter, at least one hydroxy acid, and combinations thereof.

15. The cosmetic composition according to claim 14, wherein the active compound is selected from the group consisting of alpha, beta or polyhydroxy acids selected from the group consisting of lactic acid, glycolic acid, salicylic acid, malic acid, tartaric acid, citric acid, mandelic acid, lactobionic acid, gluconolactone, galactose, and combinations thereof.

16. The cosmetic composition according to claim 1, wherein the carrier system comprises a glycol or combination of glycols selected from the group consisting of: propylene glycol, dipropylene glycol, pentylene glycol, hexylene glycol, ethoxydiglycol, a combination of propylene glycol and ethoxydiglycol; a combination of pentylene glycol, hexylene glycol and dipropylene glycol; and a combination of dipropylene glycol and hexylene glycol.

17. A cosmetic composition comprising:
(a) a metal chelation system comprising:
i. ferulic acid; and
ii. one or a combination of phytic acid and chlorogenic acid;
(b) ascorbic acid; and
(c) a carrier system comprising a water phase and one more additional ingredients, each present in the water phase or in a second phase, the one or more additional ingredients including one or more of a glycol, a humectant, a preservative, a surfactant, a thickener, an active compound, or a combination thereof,
wherein the cosmetic composition is one of (i) a single phase solution comprising in the water phase a glycol or combination of glycols selected from the group consisting of: propylene glycol, dipropylene glycol, pentylene glycol, hexylene glycol, ethoxydiglycol, a combination of propylene glycol and ethoxydiglycol; a combination of pentylene glycol, hexylene glycol and dipropylene glycol; and a combination of dipropylene glycol and hexylene glycol, (ii) a multi-phase solution comprising at least one additional phase, or (iii) an emulsion comprising an oily phase,
wherein the cosmetic composition demonstrates removal of free metal ions in material contacted with the cosmetic composition, the removal of free metal ions in a range from at least about 40% and up to about 99%.

18. The cosmetic composition according to claim 17, wherein: ferulic acid is present in a range from about 0.2% to about 5%; phytic acid is present in a range from about 0.25% to about 10%; and ascorbic acid is present in a range from about 5% to about 30%, all amounts by weight, based on the weight of the composition

19. A method for providing a cosmetic composition, the method comprising:
(a) providing a metal chelation system comprising:
i. ferulic acid; and
ii. phytic acid;
(b) providing ascorbic acid;
(c) providing a carrier system comprising:
(i) a water phase; and
(ii) one or more additional ingredients includes one or more of a glycol, a humectant, a preservative, a surfactant, a thickener, an active compound, or a combination thereof; and
(iii) optionally one or more additives emulsifiers, emollients, fragrances, pH adjusters, other cosmetically acceptable additives, or a combination thereof; and
(d) combining the metal chelation system and the ascorbic acid in the water phase of the carrier system with the one or more additional ingredients and optional one or more additives to provide a cosmetic composition wherein each of the acids in the metal chelation system is solubilized in the cosmetic composition.

20. The cosmetic composition according to claim 19, wherein the carrier system comprises a glycol or combination of glycols selected from the group consisting of: propylene glycol, dipropylene glycol, pentylene glycol, hexylene glycol, ethoxydiglycol, a combination of propylene glycol and ethoxydiglycol; a combination of pentylene glycol, hexylene glycol and dipropylene glycol; and a combination of dipropylene glycol and hexylene glycol.
